(19) United States
(12) Patent Application Publication
(10) Pub. No.: US 2014/0274642 A1
(43) Pub. Date: Sep. 18, 2014

(54) METHODS FOR MANUFACTURING DISPOSABLE ABSORBENT REFASTENABLE ARTICLES COMPRISING FOLDED FASTENING TAB MEMBERS

(71) Applicant: The Procter & Gamble Company, Cincinnati, OH (US)

(72) Inventors: Gary Dean LaVon, Liberty Township, OH (US); Bret Darren Seitz, West Chester, OH (US); Mark James Kline, Okeana, OH (US); Jeromy Thomas Raycheck, South Lebanon, OH (US); Jacob Alan Barnhorst, Deerfield Towship, OH (US)

(73) Assignee: The Procter & Gamble Company, Cincinnati, OH (US)

(21) Appl. No.: 14/205,630

(22) Filed: Mar. 12, 2014

Related U.S. Application Data
(60) Provisional application No. 61/787,332, filed on Mar. 15, 2013.

(51) Int. Cl. A61F 13/15

(52) U.S. Cl. CPC A61F 13/15699
USPC 493/346

(57) ABSTRACT

A method for making a refastenable article may comprise the steps of: (1) conveying a first belt web comprising a first nonwoven layer and a first pre-strained elastic element along a machine direction; (2) conveying a second belt web comprising a second nonwoven layer and a second pre-strained elastic element along a machine direction; (3) conveying a continuous fastening web comprising a first fastening tab member along the machine direction; (4) folding the first fastening tab member; (5) cutting the fastening web into discrete webs; (6) rotating the discrete fastening web; (7) directly or refastenably joining the discrete fastening web to one of the belt webs; (8) joining the first and second belt webs to a chassis comprising a core; (9) folding the article over a lateral axis, such that the first and second belt webs generally overlap; (10) refastenably joining the first and second belt webs; and (11) cutting through the discrete fastening web to form multiple refastenable absorbent articles.

### CROSS REFERENCE TO RELATED APPLICATION

This application claims the benefit of U.S. Patent Application Ser. No. 61/787,332, filed on Mar. 15, 2013, the entire disclosure of which is hereby incorporated by reference.

### BACKGROUND

The present disclosure reveals improvements to the structural design and process for making of disposable absorbent pants having a belt configuration comprising fastening components for forming a refastenable feature at high speeds manufacturability.

As background, disposable absorbent articles in pant form may comprise an elasticized belt that encircles the wearer's waist and may form the waist edge about the entire pant. A central chassis may be joined to the elasticized belt, usually on the inside thereof, with its ends disposed at locations in the front and rear waist regions somewhat below the waist edges of the elasticized belt. This configuration is sometimes known as a “belt” or “balloon” configuration (hereinafter, “belt” configuration). An example of this type of configuration is currently manufactured and sold in Asia by The Procter & Gamble Company under the trademark PAMPERS, and also by Unicharm Corporation under the trademark MAMY POKO.

For purposes of ease of use it may be desired that the pant have a refastenable feature for enabling ease of opening and reclosing of the belt-like structure. This may be accomplished by incorporating for example mechanical fastening elements into the structure of the absorbent article as part of the belt-like structure, the central chassis, other elements of the article and/or combinations thereof. The refastenable feature may help maintain and/or establish the leg openings and the waist opening. Although some currently manufactured belt configuration pants include refastenable features they may be considered to be too complex to produce—especially at high production speeds, and too complex to use. And, some may be considered to have a rough or unfinished appearance.

The present disclosure reveals improvements to the structural design and process for making of disposable absorbent pants having a belt configuration comprising fastening components for forming a refastenable feature compatible with high speed manufacturing.

Particularly, the present disclosure reveals advantages of folding the fastening tabs. For instance, a folded fastening tab (e.g., 210) enables a narrower footprint of the absorbent article via an increased pathlength of the fastening tab's carrier layer and hooks because the extended carrier layer acts as a folded belt. The narrow footprint makes for a neater looking product. Also, this helps drive smaller packaging and portability and convenience. Further, this reduces the need to fold or tuck the belt, which is very complex given the direction of the product during manufacture.

Another advantage of folding the fastening tab is that it can be made of a different more expensive, higher stretch material than the rest of the belt. This enables the belt to be lower stretch or nonelastic. The fastening tab can be a much higher basis weight material than the belt, which is cost effective and which improves fastening performance by providing a cushion for the fastening elements, which may be made of a stiffer material. The more expensive material (that the fastening tab could be made of) could be a film versus a nonwoven to enable alternative forms of fastening like adhesives, cohesive, and the like. Another expensive material for fabricating the fastening tab may include a film-strand laminate.

Further, folding the fastening tab enables disposition of the fastening elements more on the front or back on belts without folding either of the belts. For example, the fastening tabs may be back-biased on larger sizes for older wearers to avoid having the child remove or open the fastener. Alternatively, the fastening tabs may be front-biased for younger kids who lay on their backs to get changed.

### DESCRIPTION OF THE DRAWINGS

FIG. 1 is simplified perspective view of a disposable absorbent pant;

FIG. 2 is a simplified plan view of a precursor structure of a disposable absorbent pant, shown with inner or wearer-facing surfaces upward;

FIGS. 3-5 are simplified, schematic cross-section views of portions of a multilayer web comprising fastening components.

FIG. 6 is a plan view of a multilayer web of FIG. 3 and applied chassis structures illustrating a method for manufacturing pant structures;

FIG. 7 is a plan view of a multilayer web of FIG. 4 and applied chassis structures illustrating a method for manufacturing pant structures;

FIG. 8 is a plan view of a multilayer web of FIG. 5 and applied chassis structures illustrating a method for manufacturing pant structures;

### DEFINITIONS

“Fastening component” refers to the fastening elements that define an area of refastenable attachment. The fastening components enable refastening of the absorbent article to reconfigure the waist and leg openings into a closed configuration until the fastening components are separated. A fastening component may comprise of one or more refastenable fastening elements, e.g., hooks, loops, bulbs, mushrooms, arrowheads, balls on stems, buttons, snaps, refastenable cohesives, selective refastenable adhesives, etc. A fastening component may be opened and subsequently re-closed, reliably, without destroying the fastening component. A fastening component comprises those elements of a fastening system that form the area of attachment via direct surface-to-surface contact forming a refastenable closure. For the purpose of clarity, surface-to-surface contact encompasses contact between a surface of a hook material and a surface of a loop material, for example. For instance, a tab member joined to a backsheet would not be a fastening member as discussed. The fastening component may be the hooks or the loops that are joined to the tab and connect with the other fastening components or a portion of an absorbent article.

“Cross direction” (CD)—with respect to the making of a nonwoven web material, the nonwoven material itself, a laminate thereof, or an article in which the material is a component, refers to the direction along the material substantially perpendicular to the direction of forward travel of the material through the manufacturing line in which the material and/or article is manufactured.

“Elastic” or “elastomeric” refers to the property of an extensible material (or a composite of multiple materials) that can extend, without substantial rupture or breakage, to a strain of 100%, with a set less than or equal to 10% of the elongation. An elastic material is considered elastically extensible.

“Machine direction” (MD)—with respect to the making of a nonwoven web material, the nonwoven material itself, a laminate thereof, or an article in which the material is a component, refers to the direction along the material substantially parallel to the direction of forward travel of the material through the manufacturing line in which the material and/or article is manufactured.

“Lateral”—with respect to a pant and its wearer, refers to the direction generally perpendicular with the wearer's standing height, or the horizontal direction when the wearer is standing. “Lateral” is also the direction generally perpendicular to a line extending from the midpoint of the front waist edge to the midpoint of the rear waist edge.

“Longitudinal”—with respect to a pant and its wearer, refers to the direction generally parallel with the wearer's standing height, or the vertical direction when the wearer is standing. “Longitudinal” is also the direction generally parallel to a line extending from the midpoint of the front waist edge to the midpoint of the rear waist edge.

“Pant” (also referred to as “disposable training pant,” “training pant,” “incontinence pant,” and “pull-on pant-type diaper”) refers to disposable absorbent articles having a continuous perimeter waist opening and continuous perimeter leg openings designed for infant, child, or adult wearers (hereafter “wearer”). A pant may be configured with a continuous or closed waist opening and at least one continuous or closed leg opening prior to the pant being applied to the wearer. A pant may be pre-formed by any suitable technique including, but not limited to, joining together portions of the absorbent article using any refastenable and/or permanent closure member(s) (e.g., seams, heat bonds, pressure welds, adhesives, cohesive bonds, mechanical fasteners, etc.). A pant may be preformed anywhere along its circumference in the waist region (e.g., side fastened, front waist fastened, rear waist fastened). Example pants and pant configurations are disclosed in U.S. Pat. Nos. 5,246,433, 5,569,234, 6,120,487, 6,120,489, 4,940,464, 5,092,861, 5,897,545, 5,957,908, and U.S. Pat. Publ. No. 2003/0233082.

“Side edge seam” refers to a given side edge wherein a portion of the side edge, or region adjacent the side edge, in the front waist region is joined to a portion of the same side edge, or region adjacent the side edge, in the rear waist region to define closed, encircled leg openings and a closed waist opening.

FIG. 1 is a general simplified perspective depiction of a disposable absorbent pant 10 having a belt configuration. Pant 10 may include a central chassis 20 and an elasticized belt 30. Elasticized belt 30 may be elastically extensible in the lateral direction, providing elastic stretchability for ease of donning, and a snug and comfortable fit following donning Central chassis 20 may include a wearer-facing, liquid permeable topsheet (not specifically shown in FIG. 1), an outer- or garment-facing backsheet (not specifically shown in FIG. 1) and an absorbent core (not specifically shown in FIG. 1) sandwiched or enveloped between the topsheet and backsheet. A pair of laterally opposing, longitudinally extending barrier cuffs 25 also may be included with the central chassis in a crotch region thereof, disposed adjacent to the topsheet. Generally the central chassis and barrier cuffs may have any construction and components, including leg cuff structures, suitable for disposable diapers, training pants, and adult incontinence pants, such as, but not limited to, those described in U.S. provisional patent application No. 61/480,663 and application(s) claiming priority thereto. Elasticized belt 30 may have a front portion 31 and a rear portion 32. Front and rear portions 31, 32 may be joined together at respective left and right side edge seams 33l, 33r. Elasticized belt 30 may form front and rear waist edges 11, 12 defining waist opening 15, and at least portions of left and right leg opening edges 13l, 13r of the pant 10. The disposable absorbent pant 10, and more particularly, the elasticized belt 30 may comprise fastening components 200l and 200r for creating a refastenable feature.

FIG. 2 is a simplified plan view of the precursor structure of the pant 10 shown in FIG. 1, shown prior to joining of front and rear portions 31, 32 along their respective side edges 34l, 35l and 34r, 35r. Front region 31a, including front portion 31, and rear region 32a, including rear portion 32, may each include anywhere from 25 percent to 40 percent of the overall longitudinal length of the precursor structure; correspondingly, a crotch region 45 may include anywhere from 20 percent to 50 percent of the overall longitudinal length of the precursor structure, with at least a portion thereof lying at lateral axis 110. To form pant 10, the precursor structure may be folded along lateral axis 110 to bring front and rear regions 31a, 32a, and front and rear portions 31, 32 together such that their side edges 34l, 35l and 34r, 35r, respectively, may be joined at side edge seams 33l, 33r (as shown in FIG. 1). The embodiment shown in FIG. 2 comprises fastening elements 201-204 that may be refastenably joined together. Particularly, fastening elements 201 and 203 may be hook elements that join with fastening elements 202 and 204, respectively. Fastening elements 201 and 203 are shown on an exterior surface of the elasticized belt 30, but they may also be placed on an interior surface of the elasticized belt 30. Fastening elements 202 and 204 may be a discrete member of loop elements or may be an area of loop elements that is part of a nonwoven sheet lining the interior (as shown) or exterior of the elasticized belt. In another embodiment, fastening elements 201 and 203 may be loop elements and fastening elements 202 and 204 may be hook elements. It is understood that when the fastening elements 201-204 mate interior surface to interior surface of the elasticized belt 30, a flange seam is formed. But, when the fastening elements 201-204 mate interior surface to exterior surface of the elasticized belt 30, an overlap seam is formed, as illustrated in FIG. 1. The fastening elements 201-204 may be fastened during the manufacturing process and/or fastened in the package prior to use by the wearer or caregiver (i.e., the pant may be sold in “closed form”). Alternatively, the pant may be sold in “open form,” where the fastening elements 201-204 are not joined in the package.

Still referring to FIG. 2, one or both of front and rear portions 31, 32 may include at least a first elastic member 36, 37 disposed nearer the waist edges 11, 12 and at least a second elastic member 38, 39, disposed nearer the leg opening edges 13l, 13r. Of course, as illustrated in FIGS. 1 and 2, various other elastic members may reside between the first and second elastic members. As suggested in FIG. 2, one or a plurality of waist elastic members 36, 37 may be disposed in a substantially straight lateral orientation, and one or a plurality of leg elastic members 38, 39 may be disposed along curvilinear paths to provide hoopwise elastic stretch about the leg openings 13l, 13r (as shown in FIG. 1). For purposes of manufacturing a pant having a neat appearance as will be described below, it may be desired that leg elastic members 38, 39 terminate proximate the respective longitudinal edges 21 of chassis 20.

Elastic members 36, 37, 38 and 39 may be in the form of film or sections or strips thereof, strips, ribbons, bands, scrims or strands of circular or any other cross-section, formed in any configuration of any elastomeric material such as described in, for example, co-pending U.S. application Ser. Nos. 11/478,386 and 13/331,695, and U.S. Pat. No. 6,626,879. A suitable example is LYCRA HYFIT strands, a product of Invista, Wichita, Kans.

The elasticized belts and rear region of the pant may be in a number of configurations as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of the '065 application). As described in U.S. Ser. No. 13/764,990, filed on Feb. 12, 2013, the elastic members 37 may be assembled onto a full outer cover nonwoven, where an inner nonwoven sandwiches them. In such a configuration, the legs may be cut out. Also as described in the '990 application, the inner and outer nonwovens of approximately the same dimension may be used to sandwich the elastics to form front belts and back belts, which are laterally spaced as they travel the machine direction, and are bridged by a central chassis that may comprise a backsheet film, absorbent gelling material, a topsheet, and cuffs.

The elastic members 37 may be pre-strained in a number of configurations to create texture as described and illustrated in FIGS. 8 and 9 of U.S. Ser. No. 61/666,065, filed on Jun. 29, 2012, titled DISPOSABLE ABSORBENT REFASTENABLE PANTS AND METHODS FOR MANUFACTURING THE SAME.

In various embodiments, each of the fastening components 200 may be joined directly or indirectly to the pant 10 by any suitable methods, such as adhesive bonding, sonic bonding, pressure bonding, thermal bonding or combinations thereof, for example. Some suitable examples of fastening systems and/or the fastening components 200 are discussed in U.S. Pat. Nos. 3,848,594, 4,662,875, 4,846,815, 4,894,060, 4,946,527, 5,151,092, 5,221,274, 6,251,097, 6,669,618, 6,432,098, 7,101,359, and 7,407,468.

FIG. 3 illustrates the formation of two articles folded over a lateral axis (e.g., 110 in FIGS. 2 and 6-8) to form pants, e.g., pant 10-1 and pant 10-2, wherein only a portion of each is shown. In this embodiment, which is only one embodiment of the present disclosure, each of the pants 10-1 and 10-2 comprise front belts 30a that extend from a first side edge 34r and 34l, respectively, to a laterally opposing second side edges (not shown) and form portions of laterally opposing permanent side edge seams 208. Of course, each of the belts is continuous belt webs prior to being cut along cutline 209.

Also for each of the pants 10-1 and 10-2, the rear belts 30b extend from adjacent first side edges 35r and 35l, respectively, to an area adjacent the opposing second side edges (not shown) and do not form a portion of the laterally opposing permanent side edge seams 208. The pants 10-1 and 10-2 further comprise fastening tab members 210 disposed between the front and rear belts 30a and 30b. The fastening tab members 210 of this embodiment are permanently joined to a wearer facing surface of the front belt and comprise first and second fastening elements 202 and 204 disposed between the fastening tab member 210 and the rear belts 30b and are capable of being refastenably engaged with a wearer facing surface of the rear elasticized belts 30b. In this embodiment, the fastening tab member 210 is double folded (laterally inward, then laterally outward) such that the fastening tab members 210 comprises first folds 302a and 302b, and second folds 304a and 304b. The web making up the fastening tab members 210 may be folded prior to being joined to the elasticized belts 30 (for this embodiment, as well as the embodiments of FIGS. 4 and 5 also, and generally).

Temporary bonds 300a-c may used to control the web and/or to keep the fastening tab members 210 tight against the rear belts 30b. Temporary bonds 300a-c may each vary in bond strength (e.g., 300a may be weaker than 300b, which may be weaker than 300c) or they may all be the same strength. Temporary bonds may also be used on the webs making up the fastening tab members 210 of the embodiments of FIGS. 4 and 5, and generally. The fastening components 200 may comprise second fastening elements 201 and 203 formed in or disposed on the rear belts 30b.

In an alternative embodiment of the present disclosure (not shown), an identical embodiment to FIG. 3, except that fastening tab member 210 may be attached to the rear belt 30b and the fastening elements 202 and 204 may be capable of refastenably engaging with the front elasticized belt 30a.

FIG. 4 illustrates the formation of two articles folded over a lateral axis (e.g., 110 in FIGS. 2 and 6-8) to form pants, e.g., pant 10-1 and pant 10-2, wherein only a portion of each is shown. In this embodiment, which is only one embodiment of the present disclosure, each of the pants 10-1 and 10-2 comprise rear belts 30b that extend from a first side edge 35r and 35l, respectively, to a laterally opposing second side edges (not shown) and form portions of laterally opposing permanent side edge seams 208b. Also, for each of the pants 10-1 and 10-2, the front belts 30a extend from adjacent a first side edges 34r and 34l, respectively, to an area adjacent the opposing second side edges (not shown) and form a portion of the laterally opposing permanent side edge seams 208a. The articles further comprise fastening tab members 210a permanently joined to a wearer facing surface of the rear belts 30b. In this embodiment, the fastening tab member 210a is single folded such that the fastening tab member 210 comprises first folds 302a and 302b. The articles also comprise first fastening elements 202 and 204 disposed on fastening tab member 210b (permanently joined to a wearer facing surface of the front belts 30a) and are capable of being refastenably engaged with a wearer facing surface of the fastening tab members 210a. The fastening components 200 may comprise second fastening elements 201 and 203 formed in or disposed on the fastening tab members 210a.

In an alternative embodiment of the present disclosure (not shown), an identical embodiment to FIG. 4, except that fastening tab member 210a may be attached to front belt 30a and fastening tab member 210b may be attached to rear belt 30b.

FIG. 5 illustrates the formation of two articles folded over a lateral axis (e.g., 110 in FIGS. 2 and 6-8) to form pants, e.g., pant 10-1 and pant 10-2, wherein only a portion of each is shown. In this embodiment, which is only one embodiment of the present disclosure, each of the pants 10-1 and 10-2 comprise rear belts 30b that extend from a first side edge 35r and 35l, respectively, to a laterally opposing second side edges (not shown) and form portions of laterally opposing permanent side edge seams 208b. Also, for each of the pants 10-1 and 10-2, the front belts 30a extend from adjacent a first side edges 34r and 34l, respectively, to an area adjacent the opposing second side edges (not shown) and form a portion of the laterally opposing permanent side edge seams 208a. The articles further comprise fastening tab members 210a permanently joined to a wearer facing surface of the rear belts 30b. In this embodiment, the fastening tab member 210a is single folded such that the fastening tab member 210 comprises first folds 302a and 302b. The articles also comprise first fastening elements 202 and 204 disposed on fastening tab member 210a and are capable of being refastenably engaged with a wearer facing surface of the fastening tab member 210b (permanently joined to a wearer facing surface of the front belts 30a). The fastening components 200 may comprise second fastening elements 201 and 203 formed in or disposed on the fastening tab members 210b.

In an alternative embodiment of the present disclosure (not shown), an identical embodiment to FIG. 5, except that fastening tab member 210a may be attached to front belt 30a and fastening tab member 210b may be attached to rear belt 30b.

Referring to FIGS. 6-8, the embodiments of FIGS. 3-5 are shown in plan view, respectively. In FIGS. 6-8, elastic members (including 36-39) are not shown. And, the articles are not yet folded over a lateral axis 110 to form prefastened pants (e.g., pant 10-1 and pant 10-2), thus a continuous subassembly is formed, which becomes discrete articles once folded and cut. Fastening tab members 210 may be made in the machine direction (i.e., along lateral axis 110) as a continuous web, then cut to length to form discrete webs, turned, and joined as discrete webs to an inner surface (which will become a wearer facing surface once the discrete pant is formed) the front and/or rear belts 30a and 30b as the belts move in the machine direction. Of course, the fastening tab members are in the form of a fastening web prior to being cut along cutline 209. Alternatively, the fastening tab members may be formed into a web oriented in the cross machine direction relative to the article, cut to length, and fed onto the belts without needing to be turned. The articles may be folded along the lateral axis 110 prior to cutting along cutline 209.

Fastening tab members 210 may overlap with elastic members before and after a cut is made along cutline 209. Alternatively, as elastic members are cut along cutline 209, the elastic members may snap outside of the area occupied by the fastening tab members 210.

In some embodiments, fastening tab members 210 may be joined to the front and/or rear belts 30a and 30b prior to joining the central chassis 20 to the belts.

For each of the embodiments illustrated by FIGS. 3-8, as well as the disclosed alternate embodiments of these Figs., it should be understood that the belts 30a and 30b may be formed in accordance with the disclosure of U.S. 61/646,999, filed on May 15, 2012. And, further, that the methodologies disclosed by U.S. Ser. Nos. 61/647,061, 61/647,071, 61/647,078, each filed on May 15, 2012, may be used, as well. And, the stress, strain, and spacing of the belt elastics may be done as disclosed in U.S. Ser. No. 13/764,990, filed Feb. 12, 2013.

Also, for each of the embodiments illustrated by FIGS. 3-8, as well as the disclosed alternate embodiments of these Figs., it should be understood that these articles may comprise the cuffs disclosed in U.S. Ser. No. 13/457,521, filed Apr. 27, 2012, and may have graphics in accordance with U.S. Serial Nos. 61/646,953 and 61/646,979, each filed on May 15, 2012. And, it may be desirable to use the hot air seaming processes, as well as the article forming processes disclosed in U.S. Pat. No. 6,248,195 and U.S. Ser. Nos. 12/795,021, 13/401,907, and 13/402,056 for seaming and forming the refastenable pants disclosed in each of the embodiments illustrated by FIGS. 3-8, as well as the disclosed alternate embodiments of these Figs. as these embodiments are particularly good for producing in excess of 1000 articles per minute due to their simplicity. The majority of the article elements including elastic elements are formed continuously in the machine direction. In addition, such a process enables introduction and bonding of refastenable elements to these continuous machine direction sub structures at high speed while maintaining accuracy of placement and ensuring integrity which is key to the products functionality and appearance.

With regard to processing, each of the embodiments illustrated by FIGS. 3-8, as well as the disclosed alternate embodiments of these Figs., offer the advantage of a folded fastener tab member. And, because these embodiments offer a permanent side seam, thinner materials can be used for the fastening component. Even elastomeric materials can be used for the fastening components.

All patents and patent applications (including any patents which issue thereon) referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” All documents cited in the Detailed Description of the Invention are, in relevant part, incorporated herein by reference; the citation of any document is not to be construed as an admission that it is prior art with respect to the present disclosure. To the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is, therefore, intended that the scope of the invention is limited only by the appended claims and equivalents thereof

What is claimed is:

1. A method for manufacturing disposable absorbent articles, comprising the steps of:
(1) conveying a first belt web comprising a first nonwoven layer and a first pre-strained elastic element along a machine direction;
(2) conveying a second belt web comprising a second nonwoven layer and a second pre-strained elastic element along a machine direction;
(3) conveying a continuous fastening web comprising a first fastening tab member along the machine direction,
(4) folding the first fastening tab member;
(5) cutting the fastening web into discrete webs;
(6) rotating the discrete fastening web;
(7) directly or refastenably joining the discrete fastening web to one of the belt webs;
(8) joining the first and second belt webs to a chassis comprising a core;
(9) folding the article over a lateral axis, such that the first and second belt webs generally overlap;
(10) refastenably joining the first and second belt webs; and
(11) cutting through the discrete fastening web to form multiple refastenable absorbent articles.

2. The method of claim 1, wherein the discrete fastening web is rotated between 45 and 135 degrees.

3. The method of claim 1, wherein the discrete fastening web is disposed between the first and second belt webs.

4. The method of claim 1, wherein the first fastening tab member comprises a fastening element.

5. The method of claim 1, wherein the fastening element is a plurality of hooks.

6. The method of claim 1, wherein the first fastening tab member is joined to the first belt web and wherein the first fastening element is joined to the second belt web, and wherein the first fastening element is refastenably joined to the first fastening tab member.

7. The method of claim 1, wherein the first fastening tab member is folded and wherein a portion of a second fastening tab member is disposed within a fold of the first fastening tab member.

8. The method of claim 1, wherein the first fastening tab member is refastenably joined to a first fastening element.

9. The method of claim 1, wherein a first fastening element is refastenably joined to at least one of the first and second belts.

10. The method of claim 1, wherein a first permanent side seam is formed between the first fastening tab member and one of the first and second belt webs, and a second permanent side seam is formed between a second fastening tab member and one of the first and second belt webs prior to directly or refastenably joining the first fastening tab member to the second fastening tab member.

11. The method of claim 1, further comprising the step of forming a permanent side seam between at least one of: a) a first fastening tab member and at least one of the first and second belt webs, b) the first fastening tab member and both of the first and second belt webs, c) the first fastening tab member and a second fastening tab member.

12. The method of claim 1, wherein at least one of the fastening tab members is double folded.

13. The method of claim 1, wherein temporary bonds are placed between one or more folded portions of the fastening tab member.

14. The method of claim 1, wherein one or more temporary bonds are placed between the discrete fastening web and at least one of the belt webs.

15. A method for manufacturing disposable absorbent articles, comprising the steps of:
(1) conveying an outer nonwoven web comprising a first nonwoven layer and a first pre-strained elastic element along a machine direction;
(2) conveying an inner nonwoven web along a machine direction;
(3) conveying a continuous fastening web comprising a first fastening tab member along the machine direction,
(4) folding the first fastening tab member;
(5) cutting the fastening web into discrete webs;
(6) rotating the discrete fastening web;
(7) directly or refastenably joining the discrete fastening web to one of the inner or outer nonwoven webs;
(8) joining the inner and outer webs to a chassis comprising a core;
(9) folding the article over a lateral axis, such that the inner nonwoven web generally overlaps itself;
(10) refastenably joining the inner and/or the outer nonwoven webs to form waist and leg openings; and
(11) cutting through the discrete fastening web to form multiple refastenable absorbent articles.

16. A method for manufacturing disposable absorbent articles, comprising the steps of:
(1) conveying a first belt web comprising a first nonwoven layer and a first pre-strained elastic element along a machine direction;
(2) conveying a second belt web comprising a second nonwoven layer and a second pre-strained elastic element along a machine direction;
(3) conveying a first continuous fastening web comprising a first fastening tab member along the machine direction;
(4) conveying a second continuous fastening web along the machine direction;
(5) cutting the first fastening web;
(6) cutting the second fastening web;
(7) folding at least one of the first and second fastening webs;
(8) permanently joining the first fastening web to one of the first and second belt webs;
(9) permanently joining the second fastening web to one of the first and second belt webs;
(10) refastenably joining the first fastening web to the second fastening web
(11) joining the first and second belt webs with a center chassis comprising a core;
(12) folding the article over a lateral axis, such that the first and second belt webs generally overlap;
(13) cutting through the first and second discrete fastening webs to form multiple refastenable absorbent articles.

17. The method of claim 16, wherein the first fastening web is folded and the second fastening web comprises fastening elements on an inner surface (the eventual wearer facing surface).

18. The method of claim 17, wherein the fastening elements refastenably engage an inner surface (the eventual wearer facing surface) of the first fastening web.

19. The method of claim 16, wherein the second fastening web is folded and comprises fastening elements on an inner surface (the eventual wearer facing surface).

20. The method of claim 19, wherein the fastening elements refastenably engage an inner surface (the eventual wearer facing surface) of the first fastening web.
